(19) United States
(12) Patent Application Publication
(10) Pub. No.: US 2016/0120393 A1
(43) Pub. Date: May 5, 2016

(54) LAPAROSCOPIC VIEWING SYSTEM

(71) Applicant: Thomas Frederick HELMSWORTH, Muscatine, IA (US)

(72) Inventor: Thomas Frederick HELMSWORTH, Muscatine, IA (US)

(21) Appl. No.: 14/529,244

(22) Filed: Oct. 31, 2014

(51) Int. Cl. A61B 1/00; A61B 1/313

(52) U.S. Cl. CPC A61B 1/00048; A61B 1/3132; A61B 1/00193; A61B 1/00016; A61B 1/00009

(57) ABSTRACT

A laparoscopic viewing system is provided. The laparoscopic viewing system embodies a viewing device for heads-up three dimensional viewing of a laparoscopic surgical field. The viewing device may include a head-mount framing, a transparent light shield and two prism and screen sets. The head-mount framing may provide a brace arm extending off a front portion thereof for supporting the light shield a predetermined distance from the front portion. Each prism and screen set may be disposed on the light shield so as to be a predetermined distance from each eye of the wearer of the device, wherein a screen of each set is configured to produce video images viewable through its corresponding prism of the same set. The viewing system may further provide a wireless transmitter configured to reproduce visual images on each screen, wherein the visual images are transmitted from a stereo laparoscopic camera by way of the wireless transmitter.

### BACKGROUND OF THE INVENTION

The present invention relates to laparoscopic surgery and, more particularly, to a wearable device and system for heads-up three dimensional viewing of the laparoscopic surgical field.

Laparoscopic surgery is the use of a laparoscope, a long fiber optic cable system, which allows viewing on at least one display screen of a patient's affected area by snaking the cable from a more distant, but more easily accessible location. During surgery, the surgeon's hands manipulate laparoscopic instruments that remotely interact with the patient's tissue while the surgeon's eyes constantly monitor the at least one display screen.

While laparoscopic surgery has advantages compared to traditional, open surgery, the laparoscopic procedure is more difficult from the surgeon's perspective because of their poor depth perception, the constant monitoring of the at least one display screen, the limited range of motion due to the additional bulk of the laparoscopic instruments interposed between the surgeon and the surgical field. The restricted vision and the limited working area can cause neck and body positional strain because of the constant viewing of the at least one display screen. Additionally, the bulk of the laparoscopic instruments and the remote nature of laparoscopic surgery militate that the surgeon and their staff work remotely from a work station, as there is no current system or device that provides an advantage when working near the laparoscopic surgical field, such as a system of device that facilitates heads-up viewing of the surgeon's surroundings so as to allow for a view of “the whole picture” or at least fuller picture.

As can be seen, there is a need for a wearable device and system for heads-up three dimensional viewing of the laparoscopic surgical field.

### SUMMARY OF THE INVENTION

In one aspect of the present invention, a laparoscopic viewing device for a human wearer, comprises: a head-mount framing comprising a crown body for securely receiving a portion of the top of a human wearer's head, to be supported thereon; a transparent light shield connected to the head-mounting framing so that the light shield is disposed a predetermined distance from a front portion of the crown body; and two prism and screen sets provided along the light shield, wherein each set comprises a prism and a screen, wherein each prism has a viewing surface and a field of regard, wherein each screen is disposed within the field of regard, and wherein each viewing surface is disposed a predetermined distance from each eye of the wearer of the viewing device whereby facilitating heads-up three dimensional viewing of the wearer's surrounding so as to reduce neck positional strain.

In another aspect of the present invention, a laparoscopic viewing system for heads-up three dimensional viewing of the laparoscopic surgical field, comprises: a viewing device head-mount framing comprising a crown body for securely receiving a portion of the top of a human wearer's head, to be supported thereon; a transparent light shield connected to the head-mounting framing so that the light shield is disposed a predetermined distance from a front portion of the crown body; and two prism and screen sets provided along the light shield, wherein each set comprises a prism and a screen, wherein each prism has a viewing surface and a field of regard, wherein each screen is disposed within the field of regard, and wherein each viewing surface is disposed a predetermined distance from each eye of the wearer of the viewing device; a stereo laparoscopic camera configured to produce a plurality of separable left video images and right video images; and a wireless transmitter electronically connected to the stereo laparoscopic camera and the two prism and screen sets so as to reproduce the plurality of left video images and right video images separately on each screen whereby facilitating heads-up three dimensional viewing of the laparoscopic surgical field so as to reduce neck positional strain.

These and other features, aspects and advantages of the present invention will become better understood with reference to the following drawings, description and claims.

### BRIEF DESCRIPTION OF THE DRAWINGS

FIG. 1 is a detailed perspective view of an exemplary embodiment of the present invention;

FIG. 2 is a front view of an exemplary embodiment of the present invention, shown in use;

FIG. 3 is a front/schematic view of an exemplary embodiment of the present invention;

FIG. 4 is a side detail view of an exemplary embodiment of the present invention, shown in use; and

FIG. 5 is a side detail view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a viewing system embodying a viewing device for heads-up three dimensional viewing of a laparoscopic surgical field. The viewing device may include a head-mount framing, a transparent light shield and two prism and screen sets. The head-mount framing may provide a brace arm extending off a front portion thereof for supporting the light shield a predetermined distance from the front portion. Each prism and screen set may be disposed on the light shield so as to be a predetermined distance from each eye of the wearer of the device, wherein a screen of each set is configured to produce video images viewable through its corresponding prism of the same set. The viewing system may further provide a wireless transmitter configured to reproduce visual images on each screen, wherein the visual images are transmitted from a stereo laparoscopic camera by way of the wireless transmitter.

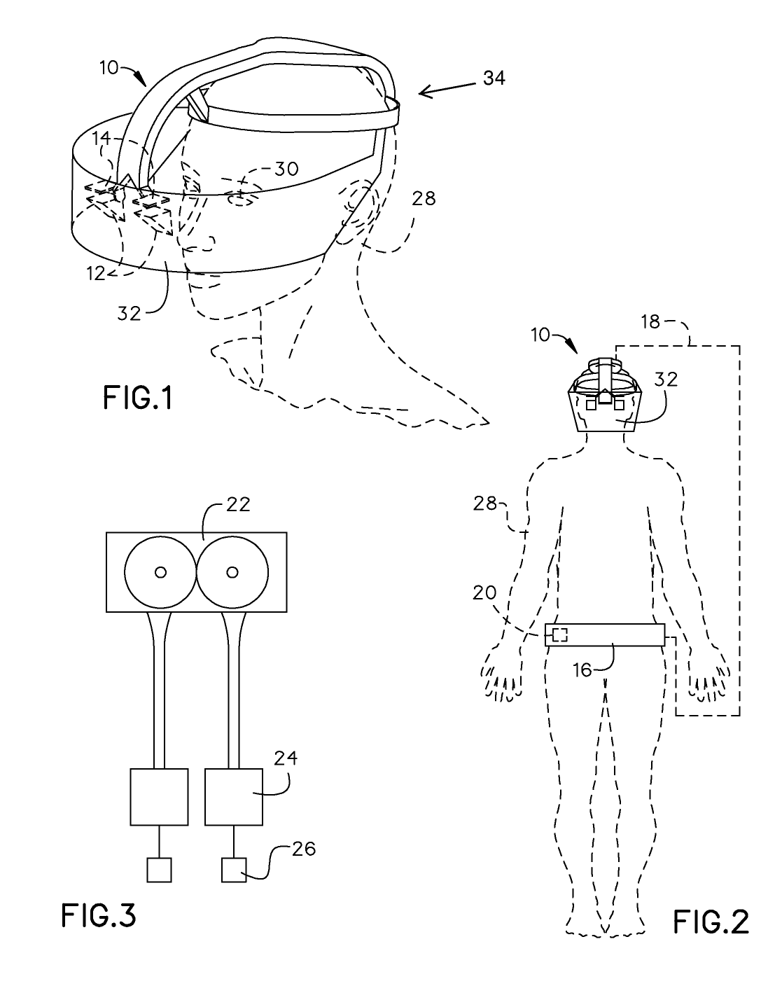

FIGS. 1 through 5 illustrate a viewing system embodying a viewing device 10 of the present invention for heads-up three dimensional viewing of a laparoscopic surgical field.

The viewing device 10 may provide an elongated light shield 32 attached to a head-mount framing 34. The elongated light shield 32 may be made of a transparent or otherwise see-through material. The elongated light shield 32 may be curved, form right angles or otherwise be suitable for attaching to the head-mount framing 34 and facilitate the viewing capabilities disclosed herein. The elongated light shield 32 may have a length spanning from a left edge to a right edge, and also having an inner surface along said length.

Two prisms 12, a left prism 12 and a right prism 12, may be affixed to the inner surface so that each prism 12 is, relative to the other, approximately equidistant from opposing sides of an approximate midpoint of the length of the inner surface. Each prism 12 may have a viewing surface 13 and a field of regard 15. Each prism 12 may be adapted to displace images within the field of regard 15 so as to be viewed through the viewing surface 13.

The head-mount framing 34 may form a crown body attached to a brace arm portion. The crown body may be adapted to receive a portion of the top of a human wearer's head, to be supported thereon, much like a crown. The crown body may have a front portion, a left portion and a right portion. The front portion may be disposed near the wearer's forehead. The left portion may be disposed near a left side of the wearer's head. The right portion may be disposed near a right side of the wearer's head. The brace arm portion may extend off the front portion of the crown body so as to support the light shield 32 near its midpoint as the light shield 32 extends to connect its left edge to the left portion and its right edge to the right portion.

The viewing device 10 may be adapted so that when worn by the human wearer 28, the viewing surface 13 of the left prism 12 may be disposed on the light shield 32 a predetermined eye distance from a left eye 30, and the viewing surface 13 of the right prism 12 is approximately the predetermined eye distance from a right eye 30 of the wearer 28. The light shield 32 may provide a video screen 14 a predetermined screen distance within the field of regard 15 of each prism 12, so that there is in effect an operable left video screen 14 associated with the left prism 12 and an operable right video screen 14 associated with the right prism 12, as illustrated in FIG. 5.

The viewing system may further provide a wireless receiver 20, a video processor 24, a power source 16, a stereo laparoscopic camera 22 and a wireless transmitter 26. The power source 16 may be a battery disposed on the belt of the wearer or other suitable locations to facilitate their freedom of movement.

As part of the related laparoscopic surgery, the laparoscope for the viewing of the laparoscopic surgical field may be operated independently within the patient. The laparoscope may terminate in the stereo laparoscopic camera 22. The stereo laparoscopic camera 22 may be electronically connected to the video processor 24, which in turn is electronically connected to the wireless transmitter 26. Note, the stereo laparoscopic camera 22 may also be electronically connected to the at least one display screen. The stereo laparoscopic camera 22 may produce a left video image and a right video image. The wireless transmitter 26 and the video processor 24 may be configured to process each video image separately.

The wireless receiver 20 may be removably connected to the same belt or other suitable location as that of the power source 16, so as to be electrically connected thereto. The wireless receiver 20 may be electrically connected to the viewing device 10 by an exemplary electrical connection 18. The wireless receiver 20 may be configured to transmit the separate left video images and right video images so as the reproduce each on the left video screen 14 and the right video screen 14 respectively.

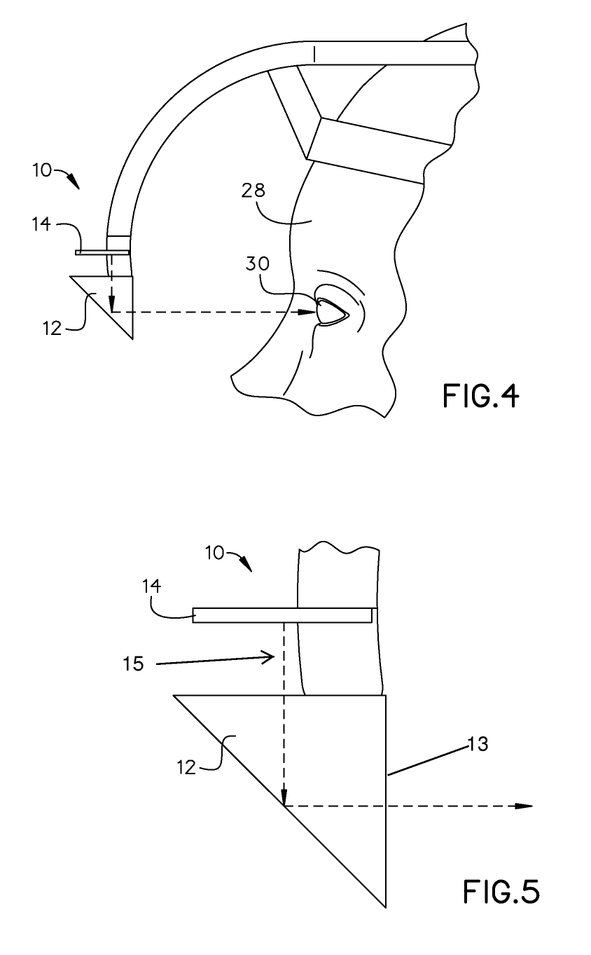

A method of using the present invention may include the following. The viewing system 10 disclosed above may be provided. A surgeon 28 may wear the viewing device 10 so that the left video image associated with the stereo laparoscopic camera 22 can be viewed by their left eye 30 looking through the viewing surface 13 of the left prism 12 on the left side of the light shield 32, as illustrated in FIGS. 4 and 5. The right video image can be seen in a similar fashion. As a result, the stereo, three-dimensional video images are directed into the surgeon's immediate field of vision so as to enable the surgeon 28 to look away from the video images to view the operative field or surrounding area and then easily return their attention to the video image. The ability to view the operative field and its environs, allows the following advantages: lessens the likelihood of neck and body positional strain of the surgeon that comes from constant viewing of the at least one display screen; and facilitates heads-up viewing of the surgeon's surroundings so as to allow for a view of “the whole picture” or at least a fuller picture, especially when working near the surgical field.

It should be understood, of course, that the foregoing relates to exemplary embodiments of the invention and that modifications may be made without departing from the spirit and scope of the invention as set forth in the following claims.

What is claimed is:

1. A laparoscopic viewing device for a human wearer, comprising:
a head-mount framing comprising a crown body for securely receiving a portion of the top of a human wearer's head, to be supported thereon;
a transparent light shield connected to the head-mounting framing so that the light shield is disposed a predetermined distance from a front portion of the crown body; and
two prism and screen sets provided along the light shield, wherein each set comprises a prism and a screen, wherein each prism has a viewing surface and a field of regard, wherein each screen is disposed within the field of regard, and wherein each viewing surface is disposed a predetermined distance from each eye of the wearer of the viewing device,
whereby facilitating heads-up three dimensional viewing of the wearer's surrounding so as to reduce neck positional strain.

2. The laparoscopic viewing device of claim 1, wherein the light shield extends from a left portion of the crown body to a right portion of the crown body.

3. The laparoscopic viewing device of claim 2, wherein the light shield curves as it extends from the left portion to the right portion of the crown body.

4. The laparoscopic viewing device of claim 1, wherein the light shield has an inner surface, and wherein the two prism and screen sets are disposed thereon.

5. The laparoscopic viewing device of claim 1, further comprising a brace arm extending off the front portion of the crown body to support the light shield.

6. A laparoscopic viewing system for heads-up three dimensional viewing of the laparoscopic surgical field, comprising:
a viewing device head-mount framing comprising a crown body for securely receiving a portion of the top of a human wearer's head, to be supported thereon; a transparent light shield connected to the head-mounting framing so that the light shield is disposed a predetermined distance from a front portion of the crown body; and two prism and screen sets provided along the light shield, wherein each set comprises a prism and a screen, wherein each prism has a viewing surface and a field of regard, wherein each screen is disposed within the field of regard, and wherein each viewing surface is disposed a predetermined distance from each eye of the wearer of the viewing device;
a stereo laparoscopic camera configured to produce a plurality of separable left video images and right video images; and
a wireless transmitter electronically connected to the stereo laparoscopic camera and the two prism and screen sets so as to reproduce the plurality of left video images and right video images separately on each screen,
whereby facilitating heads-up three dimensional viewing of the laparoscopic surgical field so as to reduce neck positional strain.

7. The laparoscopic viewing system of claim 6, further including providing a video processor electronically interconnecting the stereo laparoscopic camera.

8. The laparoscopic viewing system of claim 7, further including providing a wireless receiver electronically interconnecting the wireless transmitter and each screen, wherein the wireless receiver is configured to reproduce the plurality of left video images and right video images separately on each screen.

9. The laparoscopic viewing system of claim 7, further including providing a power source electrically connected to the wireless receiver.

10. The laparoscopic viewing system of claim 7, further including providing a belt for carrying the power source and the wireless receiver, wherein the belt for facilitating the wearing of further components of the laparoscopic viewing system, thereby reducing the limitations laparoscopic instruments impose on the working area so as to reduce neck and body positional strain.
